(19) United States
(12) Patent Application Publication
(10) Pub. No.: US 2008/0154105 A1
(43) Pub. Date: Jun. 26, 2008

(54) Electronic Signal Filtering System Suitable for Medical Device and Other Usage

(76) Inventor: Charles LeMay, Portsmouth, NH (US)

Correspondence Address: JACK SCHWARTZ & ASSOCIATES, 1350 BROADWAY, SUITE 1510, NEW YORK, NY, 10018, US

(21) Appl. No.: 11/962,342

(22) Filed: Dec. 21, 2007

Related U.S. Application Data
(60) Provisional application No. 60/871,221, filed on Dec. 21, 2006.

(51) Int. Cl. A61B 5/1455

(52) U.S. Cl. 600/330

(57) ABSTRACT

A switched filter signal processing system includes an input terminal for receiving an input signal conveying first signal information in a first time phase and second signal information in a different second time phase. Desired information represents the difference between the first and second signal information. A multiplexed switch filter filters the input signal in the first phase with a first filter to obtain the first signal information and filters the input signal in the different second time phase with a second filter to obtain the second signal information. The system also includes a common filter component, which is shared by the first and second filter, and respective second filter components for the first and second filters. A controller controls the multiplexed switch filter to couple the common filter component to the second filter component of said first filter in said first time phase and to couple the common filter component to the second filter component of the second filter in the second time phase.

This is a Non-Provisional application of U.S. Provisional Application Ser. No. 60/871,221 Filed Dec. 21, 2006.

### FIELD OF THE INVENTION

The present invention relates to switched filters, and in particular to an electronic signal filtering signal for medical or other devices.

### BACKGROUND OF THE INVENTION

Electronic signal filtering systems are sometimes sampled systems and often sampled and digitized systems. Typically, analog signals are sampled and digitized using an analog-to-digital converter (ADC). In order to prevent artifacts due to high frequency components of the signal from appearing in the sampled signal, termed aliasing, the input signal is filtered before sampling and digitization. Such filters are termed anti-aliasing filters and operate to eliminate or reduce the high frequency components of the input signal before sampling and digitization. Normally, the anti-aliasing filter provides significant attenuation at and above the Nyquist frequency of the system, which is ½ the sampling frequency. In addition, the anti-aliasing filter has a passband which is sufficiently wide to pass all frequencies-of-interest in the input signal. This, in turn, limits the sampling frequency to be at least twice the upper frequency-of-interest. However, higher sampling frequencies require higher power consumption and higher circuit cost due to the requirement for higher speed electronic components.

Some filtering systems process signals having signal information present in different time phases. For example, a system for monitoring blood oxygen saturation (SpO₂) processes a data signal having four sequential time phases. During a first time phase, a combination of ambient light and red light, typically produced by a red light emitting diode (LED), impinges on a blood perfused portion of a patient anatomy, such as a finger. A photo-detector detects light reflecting from, or passing through the blood-perfused portion of the patient anatomy. During a second time phase, the red LED is turned off and the photo-detector detects ambient light. The difference between the signals in these two phases represents desired information. During a third time phase, a combination of ambient light and infrared (IR) light, typically produced by an IR LED, impinges on the perfused portion of the patient anatomy. During a fourth time phase, the IR LED is turned off and the photo-detector detects ambient light. The difference between the signals in these two phases represents further desired information.

FIG. 2 is a block diagram of a prior art SpO₂ monitoring system and FIG. 3 illustrates waveforms useful in understanding the operation of the prior art SpO₂ monitor illustrated in FIG. 2. In FIG. 2, a controller 30 controls the time sequencing of a red LED 210 and an IR LED 212 by providing control signals to a red drive circuit 206 and an IR drive circuit 208. FIG. 3 shows the sequencing of the red and IR LEDs 210 and 212, respectively. In the top waveform of FIG. 3, the red LED drive signal is illustrated and in the second waveform of FIG. 3, the IR LED drive signal is illustrated. During a first time phase, the red LED 210 is on and the IR LED 212 is off. During a second time phase, following the first time phase, the red LED 210 and IR LED 212 are off. During a third time phase, the IR LED 212 is on and the red LED 210 is off. During a fourth time phase, the red LED 210 and IR LED 212 are off. The time phases are substantially equal in time, with a period of one millisecond (msec).

A photo-detector 214, which in the illustrated embodiment is a photodiode, receives light reflected from, or light transmitted through, a blood perfused portion of the patient anatomy, typically a finger. During the first time phase, the photo-detector 214 receives ambient light surrounding the photo-detector 214 and light from the red LED 210. During the second time phase, the photo-detector 214 receives ambient light. Desired information related to the red LED 210 is represented by the difference between the signal from the photo-detector 214 in the first and second time phases. During the third time phase, the photo-detector 214 receives ambient light and light from the IR LED 212. During the fourth time phase, the photo-detector 214 receives ambient light. Desired information related to the IR LED 212 is represented by the difference between the signal from the photo-detector 214 in the third and fourth time phases.

An input terminal of an amplifier 202 is coupled to the photo-detector 214. The amplifier 202 represents the circuitry required to extract an electrical signal representing the light received by the photo-detector 214. One skilled in the art understands what circuitry is required, how to design and implement such circuitry, and how to interconnect the circuitry with the remainder of the circuitry illustrated in FIG. 2. An output terminal of the amplifier 202 produces a signal V1 representing the light signal received by the photo-detector 214. The third waveform of FIG. 3 represents the signal V1 produced by the amplifier 202. This signal represents the light received during the four phases, and includes relatively high frequency noise.

The output terminal of the amplifier 202 is coupled to an input terminal of a multiplexed switch filter 203. An input terminal of the filter 203 is coupled to an input terminal of an input switch 205. Respective output terminals of the input switch 205 are coupled to corresponding input terminals of a plurality of filters 203(1), 203(2), 203(3) and 203(4). Filter 203(1) is representative of the filters 203(2), 203(3) and 203(4) and is illustrated in FIG. 2 as a lowpass RC filter with a resistor R1 and capacitor C1. The respective output terminals of the filters 203(1), 203(2), 203(3) and 203(4) are coupled to corresponding input terminals of an output switch 207. An output terminal of the output switch 207 produces a filtered version V2 of the light representative signal from the photo-detector 214. The fourth waveform of FIG. 3 illustrates the signal V2. FIG. 3b illustrates a more detailed waveform of one phase of the signal V2. The filter 203 provides anti-aliasing filtering and filtering for high frequency noise.

The output terminal of the multiplexed switch filter 203 is coupled to an input terminal of a buffer amplifier 204. The output terminal of the buffer amplifier 204 is coupled to an input terminal of an analog-to-digital converter (ADC) 40. An output terminal of the ADC 40 produces digital samples representing the filtered light representative signal from the photo-detector 214. The output terminal of the ADC 40 is coupled to further circuitry (not shown) which calculates a blood oxygen saturation level from the received signal information. The output terminal of the ADC 40 is also coupled to an input terminal of the controller 30. The controller 30 controls the sequencing and power applied to the red and IR LEDs 210 and 214 in response to the signal received from the ADC 40.

The controller 30 also controls the sequencing of the input and output switches 205 and 207 of the filter 203. During the first phase, the input switch 205 couples the input signal V1 to the first filter 203(1) and the output switch 207 couples the output of the first filter 203(1) to the input of the buffer amplifier 204. During the second phase, the input switch 205 couples the input signal V1 to the second filter 203(2) and the output switch 207 couples the output of the second filter 203(2) to the input of the buffer amplifier 204. During the third phase, the input switch 205 couples the input signal V1 to the third filter 203(3) and the output switch 207 couples the output of the third filter 203(3) to the input of the buffer amplifier 204. During the fourth phase, the input switch 205 couples the input signal V1 to the fourth filter 203(4) and the output switch 207 couples the output of the fourth filter 203(4) to the input of the buffer amplifier 204.

The filtered information signals in the first, second, third and fourth time phases have information in the range of frequencies up to about 10 Hz. Low pass filters 203(1), 203(2), 203(3) and 203(4), e.g. having a passband up to around 50 Hz, are sufficient to filter out high frequency noise while retaining the desired signal information. That is, noise above 50 Hz is filtered out of the resulting filtered signal. The ADC 40 operates at a sampling rate of approximately 4 kHz. Thus, the filter passband of 50 Hz also operates as an anti-aliasing filter for frequencies beyond the Nyquist frequency of 2 kHz.

However, the filtering system of FIG. 2 includes four complete low pass filters (203(1), 203(2), 203(3) and 203(4)) and an input switch 205 and an output switch 207. A filter signal processing system which provides adequate filtering of the input signal in the respective signal time phases, while reducing the number of electronic components, and the corresponding power consumption and expense, and which solves other problems with prior art filter signal processing systems, is desirable.

### BRIEF SUMMARY OF THE INVENTION

In accordance with principles of the present invention, a switched filter signal processing system includes an input terminal for receiving an input signal conveying first signal information in a first time phase and second signal information in a different second time phase. Desired information represents the difference between the first and second signal information. A multiplexed switch filter filters the input signal in the first phase with a first filter to obtain the first signal information and filters the input signal in the different second time phase with a second filter to obtain the second signal information. The system also includes a common filter component, which is shared by the first and second filter, and respective second filter components for the first and second filters. A controller controls the multiplexed switch filter to couple the common filter component to the second filter component of said first filter in said first time phase and to couple the common filter component to the second filter component of the second filter in the second time phase.

A system according to principles of the present invention provides adequate filtering of the information in the first and second phases but requires fewer filter components. This lowers power consumption, saves component cost, and increases reliability. This permits the design and implementation of a small, low power and inexpensive system while maintaining accuracy. This is particularly advantageous for medical monitoring and/or treatment devices, such as SpO₂ monitors.

### BRIEF DESCRIPTION OF THE DRAWING

In the drawing:

FIG. 1a and FIG. 1b are block diagrams of a switched filter processing system according to principles of the present invention;

FIG. 2 is a block diagram of a prior art SpO₂ monitoring system;

FIG. 3 illustrates waveforms useful in understanding the operation of the prior art SpO₂ monitor illustrated in FIG. 2;

FIG. 4 is a block diagram of an SpO₂ monitoring system according to principles of the present invention; and

FIG. 5 illustrates waveforms useful in understanding the operation of the monitoring system of FIG. 4 according to principles of the present invention.

### DETAILED DESCRIPTION OF THE INVENTION

A processor, as used herein, operates under the control of an executable application to (a) receive information from an input information device, (b) process the information by manipulating, analyzing, modifying, converting and/or transmitting the information, and/or (c) route the information to an output information device. A processor may use, or comprise the capabilities of, a controller or microprocessor, for example. The processor may operate with a display processor or generator. A display processor or generator is a known element for generating signals representing display images or portions thereof. A processor and a display processor comprises any combination of, hardware, firmware, and/or software.

An executable application, as used herein, comprises code or machine readable instructions for conditioning the processor to implement predetermined functions, such as those of an operating system, switched filter signal processing system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

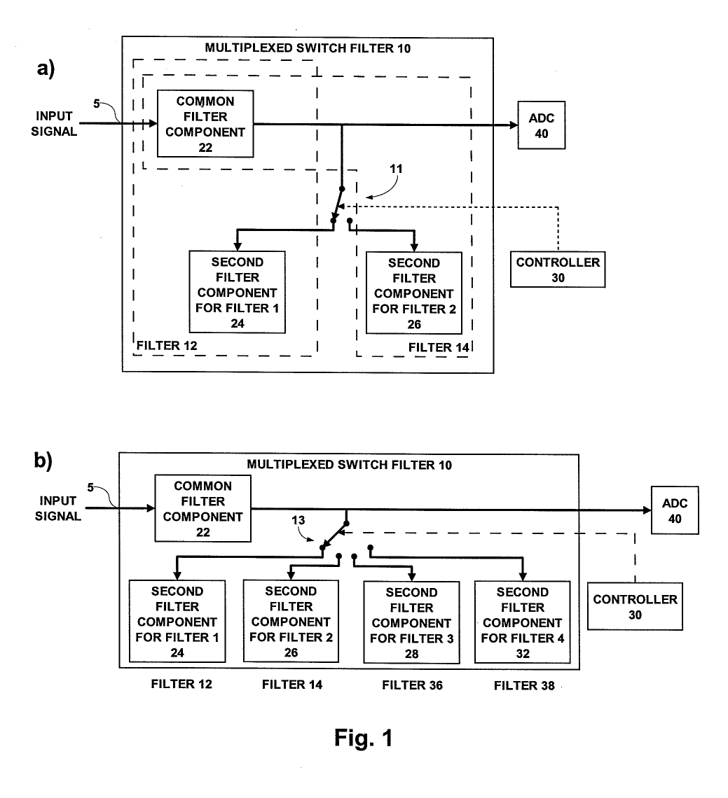

FIG. 1a and FIG. 1b are block diagrams of a switched filter processing system according to principles of the present invention. In FIG. 1a, an input terminal 5 is coupled for receiving an input signal conveying first signal information in a first time phase and second signal information in a different second time phase. Desired information represents a difference between the first and second signal information. A multiplexed switch filter 10 filters the input signal in the first time phase with a first filter 12 to obtain the first signal information and filters the input signal in the different second time phase with a second filter 14 to obtain the second signal information. A common filter component 22 is coupled to the input terminal 5. The system also includes respective second filter components 24 and 26 for the first and second filters 12 and 14, respectively. The multiplexed switch filter 10 includes a switch component 11 which operates to couple the common filter component 22 to the second filter component 24 of the first filter 12 in a first state, and to couple the common filter component 22 to the second filter component 26 of the second filter 14 in a second state. A controller 30 controls the multiplexed switch filter 10 to couple the common filter component 22 to the second filter component 24 of the first filter 12 in the first time phase and to couple the common filter component 22 to the second filter component 26 of the second filter 14 in the second time phase.

The common filter component 22 has a first electrode coupled to the input terminal 5 and a second electrode conveying the first signal information in the first time phase and the second signal information in the second time phase. The second electrode of the common filter component 22 is coupled to an analog-to-digital converter (ADC) 40. The respective second filter components 24 and 26 of the first and second filters 12 and 14, respectively, have first electrodes coupleable, through the switch component 11, to the second electrode of the common filter component 22 and second electrodes (not shown) coupled in common to a source of reference potential (ground).

The switch component 11 is coupled between the common filter component 22 and the second filter components 24 and 26 of the first and second filters 12 and 14, respectively. The switch component 11 is controlled by the controller 30 to couple the common filter component 22 to the second filter component 24 of the first filter 12 in the first time phase and to couple the common filter component 22 to the second filter component 26 of the second filter 14 in the second time phase.

The first and second filters 12 and 14 may be low pass filters. The respective filters 12 and 14 may also be (a) high pass filters and/or (b) band pass filters. The first and second filters 12 and 14, e.g. low pass, band pass, and/or high pass filters, may provide the same or different filtering characteristics.

The ADC 40 digitizes the first and second signal information, respectively. In an embodiment, the first and second signal information are represented by respective first and second voltage signals. In this embodiment, the analog-to-digital converter 40 digitizes the first and second voltage signals representing the first and second information signals, respectively.

FIG. 1b is a block diagram of another embodiment of a system according to the present invention. Those elements in FIG. 1b which are the same as those in FIG. 1a are designated by the same reference number and are not described in detail below. In FIG. 1b, the input signal further conveys third signal information in a third time phase and fourth signal information in a different fourth time phase. Further desired information represents a difference between the third and fourth signal information. The multiplexed switch filter 10 filters the input signal in the third time phase with a third filter 36 to obtain the third signal information and filters the input signal in the different fourth time phase with a fourth filter 38 to obtain the fourth signal information. In this embodiment, the common filter component 22 is shared by the first, second, third and fourth filters, 12, 14, 36 and 38. And the system further includes respective second filter components, 28 and 32, for the third and fourth filters 36 and 38, respectively.

The controller 30 controls the multiplexed switch filter 10 to couple the common component 22 to the second filter component 28 of the third filter 36 in the third time phase and to couple the common filter component 22 to the second filter component 32 of the fourth filter 38 in the fourth time phase. The second electrode of the common filter component 22 conveys the first signal information in the first time phase, the second signal information in the second time phase, the third signal information in the third phase and the fourth signal information in the fourth phase. Respective second filter components 28 and 32 of the third and fourth filters 36 and 38 have first electrodes coupleable, through a switch component 13 to the second electrode of the common filter component 22 and second electrodes (not shown) coupled in common to ground.

In this embodiment, the switch component 13 is coupled between the common filter component 22 and the second filter components 24, 26, 28 and 32, of the first, second, third and fourth filters 12, 14, 36 and 38, respectively. The switch component 13 couples the common filter component 22 to: the second filter component 24 of the first filter 12 in the first time phase; the second filter component 26 of the second filter 14 in the second time phase; the second filter component 28 of the third filter 36 in the third time phase; and the second filter component 32 of the fourth filter 38 in the fourth time phase.

In this embodiment, the third filter 36 and the fourth filter 38 may be low pass filters. The third filter 36 and fourth filter 38 may provide the same or different filtering characteristics. The third and fourth filters 36 and 38 may also be: (a) high pass filters, and/or (b) band pass filters.

The system described above and illustrated in FIG. 1 may be implemented in a medical device, and in particular in a blood oxygen level (SpO₂) monitor. In an SpO₂ monitor, the first signal information comprises a processed photo-detected signal representative of blood oxygen saturation generated in response to red LED illumination of patient anatomy and ambient light; the second signal information comprises a processed photo-detected signal representative of ambient light generated in response to switching off the red LED illumination; the third signal information comprises a processed photo-detected signal representative of blood oxygen saturation generated in response to IR LED illumination of patient anatomy and ambient light; and the fourth signal information comprises a processed photo-detected signal representative of ambient light generated in response to switching off the IR LED illumination.

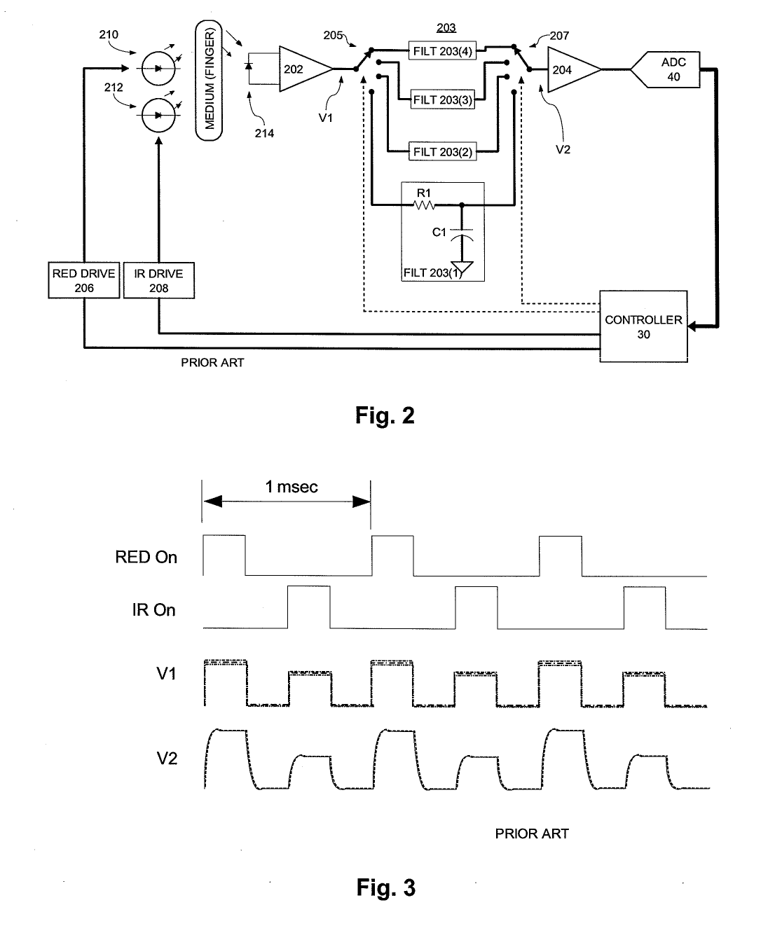

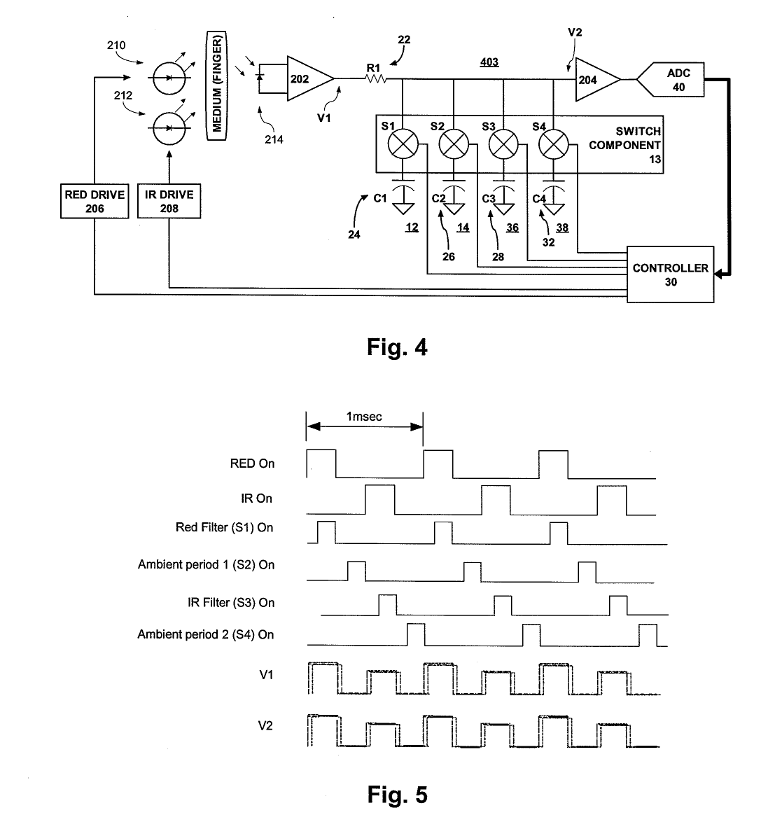

FIG. 4 is a block diagram of an SpO₂ monitor according to principles of the present invention. Elements which are the same as those illustrated in FIG. 1 and FIG. 2 are designated by the same reference number and are not described in detail below. FIG. 5 illustrates waveforms useful in understanding the operation of the SpO₂ monitor of FIG. 4.

In FIG. 4, the switched filter signal processing system is used for SpO₂ blood oxygen saturation measurement. The output terminal of the amplifier 202 generates the signal V1, and is coupled to an input terminal of a switched filter 403. The input terminal of the switched filter 403 is coupled to a first electrode of a resistor R1. A second electrode of the resistor R1 is coupled in common to first signal terminals of switches S1, S2, S3 and S4, and to an input terminal of a buffer amplifier 204. Respective second signal terminals of the switches S1, S2, S3 and S4 are coupled to corresponding first electrodes of capacitors C1, C2, C3 and C4. Respective second electrodes of the capacitors C1, C2, C3 and C4 are coupled in common to a source of reference voltage (ground). The controller 30 includes respective control output terminals, which are coupled to corresponding control input terminals of the switches S1, S2, S3 and S4. The combination of the resistor R1, switches S1, S2, S3 and S4, and capacitors C1, C2, C3 and C4 form a multiplexed switch filter 403.

In this embodiment, the common filter component 22 is the resistor R1. The respective second filter components 24, 26, 28, and 32 of the first, second, third and fourth filters, 12, 14, 36 and 38, are capacitors C1, C2, C3 and C4. The switch component 13 includes first, second, third and fourth switches, S1, S2, S3 and S4, having respective first terminals coupled in common to the second electrode of the common filter component 22 (R1), and second terminals respectively coupled to the first electrodes of the second filter components, 24, 26, 28 and 32 (C1, C2, C3 and C4), of the first, second, third and fourth filters, 12, 14, 36 and 38, respectively The controller 30 activates one switch (S1, S2, S3, S4) at a time. In FIG. 5, the top two waveforms, which illustrate the sequencing of the red and IR LEDs 210 and 212, are the same as those illustrated in FIG. 3 and are not described in detail. The third waveform illustrates the control signal for the switch S1 (FIG. 4). The switch S1 is controlled to connect the resistor R1 and the first capacitor C1 during the first time phase when the red LED 210 is on. When connected in this manner, the first filter 12 is formed from the resistor R1 and the capacitor C1. The switch S1 is controlled to isolate the capacitor C1 from the resistor R1 during the other time phases.

The fourth waveform illustrates the control signal for the switch S2 (FIG. 4). The switch S2 is controlled to connect the resistor R1 and the second capacitor C2 during the second time phase when neither the red LED 210 nor the IR LED 212 are on. When connected in this manner, the second filter 14 is formed from the resistor R1 and the capacitor C2. The switch S2 is controlled to isolate the capacitor C2 from the resistor R1 during the other time phases.

The fifth waveform illustrates the control signal for the switch S3 (FIG. 4). The switch S3 is controlled to connect the resistor R1 and the third capacitor C3 during the third time phase when the IR LED 212 is on. When connected in this manner the third filter 36 is formed from the resistor R1 and the capacitor C3. The switch S3 is controlled to isolate the capacitor C3 from the resistor R1 during the other time phases.

The sixth waveform illustrates the control signal for the switch S4 (FIG. 4). The switch S4 is controlled to connect the resistor R1 and the fourth capacitor C4 during the fourth time phase when neither the red LED 210 nor the IR LED 212 are on. When connected in this manner, the fourth filter 38 is formed from the resistor R1 and the capacitor C4. The switch S4 is controlled to isolate the capacitor C4 from resistor R1 during the other time phases.

The multiplexed switch filter 403 filters the input signal V1 in the first phase with the first filter (R1,C1) to obtain first signal information, e.g. ambient and red-LED-on light information. The multiplexed switch filter 403 filters the input signal V1 in the second time phase with the second filter (R1, C2) to obtain second signal information, e.g. ambient light information. As described above, the desired information, e.g. red-LED-on light information, represents the difference between the first signal information and the second signal information. Similarly, the multiplexed switch filter 403 filters the input signal V1 in the third phase with the third filter (R1,C3) to obtain third signal information, e.g. ambient and IR-LED-on light information. The multiplexed switch filter 403 filters the input signal V1 in the fourth time phase with the fourth filter (R1, C4) to obtain fourth signal information, e.g. ambient light information. The desired information, e.g. IR-LED-on light information, represents the difference between the third signal information and the fourth signal information. As described above, the filters 12, 14, 36 and 38, may be low pass filters. Alternatively, the filters 12, 14, 36, 38, may be: (a) high pass filters, and/or band pass filters, and they may have respectively different filter characteristics.

The filtered information signals in the first, second, third and fourth time phases have information in the range of frequencies up to about 10 Hz. A low pass filter (R1,C1; R1,C2; R1,C3 and R1,C4) having a passband up to around 50 Hz is sufficient to filter out high frequency noise while retaining the desired signal information. That is, noise above 50 Hz is filtered out of the resulting filtered signal. The ADC 40 operates at a sampling rate of approximately 4 kHz. Thus, the filter passband of 50 Hz operates as an anti-aliasing filter for frequencies beyond the Nyquist frequency of 2 kHz.

One skilled in the art understands that though the filters illustrated in FIG. 4 are RC filters, more complex or different types of filters may also be implemented in other embodiments. In addition, the characteristics of the different filters may be different in terms of passband, filter shape, etc. Further, the ADC 40 and controller 30 may be implemented by a processor operating under the control of an executable application and may implemented in hardware or software or a combination of both.

Although the invention has been described in terms of exemplary embodiments, it is not limited thereto. Rather, the appended claims should be construed broadly to include other variants and embodiments of the invention which may be made by those skilled in the art without departing from the scope and range of equivalents of the invention. This disclosure is intended to cover any adaptations or variations of the embodiments discussed herein.

What is claimed is:

1. A switched filter signal processing system, comprising:
an input terminal for receiving an input signal conveying first signal information in a first time phase and second signal information in a different second time phase and desired information represents a difference between said first and second signal information;
a multiplexed switch filter for filtering said input signal in said first time phase with a first filter to obtain said first signal information and for filtering said input signal in said different second time phase with a second filter to obtain said second signal information;
a common filter component, shared by said first and second filter, coupled to said input terminal;
respective second filter components for said first and second filters; and
a controller for controlling said multiplexed switch filter to couple said common filter component to said second filter component of said first filter in said first time phase and to couple said common filter component to said second filter component of said second filter in said second time phase.

2. A system according to claim 1 wherein:
said common filter component has a first electrode coupled to said input terminal and a second electrode conveying said first signal information in said first time phase and said second signal information in said different second time phase; and
said respective second filter components of said first and second filters have first electrodes coupleable in common to said second electrode of said common filter component and second electrodes coupled in common to a source of reference potential.

3. A system according to claim 2 wherein said multiplexed switch filter comprises a switch component coupled between said common filter component, and said second filter components of said first and second filters, respectively, to couple said common filter component to said second filter component of said first filter in said first time phase and to couple said common filter component to second filter component of said second filter in said second time phase.

4. A system according to claim 3 wherein said switch component comprises first and second switches having respective first terminals coupled in common to said second electrode of said common filter component and second terminals respectively coupled to said first electrodes of said second filter components of said first and second filters.

5. A system according to claim 4 wherein said common filter component is a resistor and said respective second filter components of said first and second filters are capacitors.

6. A system according to claim 1 wherein said first and second filter are low pass filters.

7. A system according to claim 6 wherein said first and second low pass filters may provide the same or different filtering characteristics.

8. A system according to claim 1 wherein said filter is at least one of: (a) a high pass filter and (b) a band pass filter.

9. A system according to claim 1 wherein:
said first signal information comprises a processed photo-detected signal representative of blood oxygen saturation generated in response to LED illumination of patient anatomy and ambient light; and
said second signal information comprises a processed photo-detected signal representative of ambient light generated in response to switching off said LED illumination.

10. A system according to claim 1 wherein:
said input signal further conveys third signal information in a third time phase and fourth signal information in a different fourth time phase and further desired information represents a difference between said third and fourth signal information;
said multiplexed switch filter filters said input signal in said third phase with a third filter to obtain said third signal information and filters said input signal in said different fourth time phase with a fourth filter to obtain said fourth signal information;
said common filter component is shared by said first, second, third and fourth filters;
said system further comprises respective second filter components for said third and fourth filters; and
said controller controls said multiplexed witch filter to couple said common filter component to said second filter component of said third filter in said third time phase and to couple said common filter component to said second filter component of said fourth filter in said fourth time phase.

11. A system according to claim 10 wherein:
said second electrode of said common filter component conveys said first signal information in said first time phase, said second signal information in said second time phase, said third signal information in said third time phase and said fourth signal information in said fourth time phase; and
said respective second filter components of said third and fourth filters have first electrodes coupleable in common to said second electrode of said common filter component and second electrodes coupled in common to a source of reference potential.

12. A system according to claim 11 wherein said multiplexed switch filter comprises a switch component coupled between said common filter component, and said second filter components of said first,- second, third and fourth filters, respectively, to couple said common filter component to said second filter component of said first filter in said first time phase, said second filter component of said second filter in said second time phase, said second filter component of said third filter in said third time phase and said second filter component of said fourth filter in said fourth time phase.

13. A system according to claim 12 wherein said switch component further comprises third and fourth switches having respective first terminals coupled in common to said second electrode of said common filter component and second terminals respectively coupled to said first electrodes of said second filter components of said third and fourth filters.

14. A system of claim 13 wherein said respective second filter components of said third and fourth filters are capacitors.

15. A system of claim 10 wherein said third and fourth filters are low pass filters.

16. A system according to claim 15 wherein said third and fourth low pass filters may provide the same or different filtering characteristics.

17. A system according to claim 10 wherein said third and fourth filters are at least one of: (a) a high pass filter and (b) a band pass filter.

18. A system according to claim 10 wherein:
said first signal information comprises a processed photo-detected signal representative of blood oxygen saturation generated in response to red LED illumination of patient anatomy and ambient light;
said second signal information comprises a processed photo-detected signal representative of ambient light generated in response to switching off said red LED illumination;
said third signal information comprises a processed photo-detected signal representative of blood oxygen saturation generated in response to IR LED illumination of patient anatomy and ambient light;
said fourth signal information comprises a processed photo-detected signal representative of ambient light generated in response to switching off said IR LED illumination.
